(19) United States
(12) Plant Patent Application Publication
(10) Pub. No.: US 2002/0066129 P1
(43) Pub. Date: May 30, 2002

(54) Lily plant named 'rousillon'

(76) Inventor: Cees A. van der Voort, JN Katwijk (NL)

Correspondence Address: Foley & Lardner, Washington Harbour, 3000 K Street NW, Suite 500, Washington, DC, 20007-5109, US

(21) Appl. No.: 09/521,611

(22) Filed: Mar. 9, 2000

(51) Int. Cl. A01H 5/00

(52) U.S. Cl. PLT/315

(57) ABSTRACT

A new and distinct cultivar of Lily plant named ‘Rousillon’ characterized by its erect to vertical flowers, intense purple-red color with contrasting orange pollen and stripes, spots and flakes on the inner side of the throat, and vigorous growth.

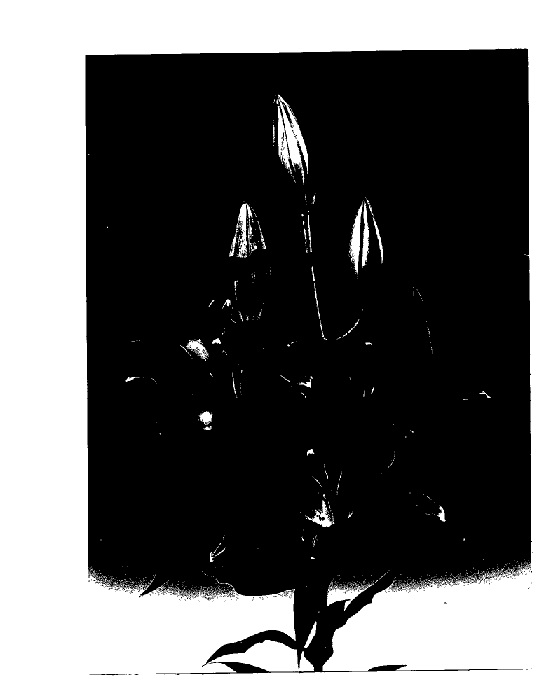

### BACKGROUND OF THE INVENTION

The present invention comprises a new and distinct cultivar of lily plant, botanically known as Lilium L., and hereinafter referred to by the cultivar name ‘Rousillon’.

‘Rousillon’ is a product of a planned breeding program which had the objective of creating new lily cultivars having many large erected flowers and vigorous growth.

‘Rousillon’ was originated from a hybridization made by the inventor in a controlled breeding program in Rijnsburg, the Netherlands in 1992. The male and female parents were unnamed seedlings of Lilium L. oriental hybrids. Both parents are proprietary cultivars used in the breeding program.

‘Rousillon’ was discovered and selected as one flowering plant within the progeny of the stated cross by the inventor, Cees A. van der Voort, in the months of May and June 1992 in a controlled environment in Rijnsburg, the Netherlands.

The first act of asexual reproduction of ‘Rousillon’ was accomplished when scales were taken from the initial selection in October 1994 in a controlled environment in Rijnsburg, the Netherlands by, or under the supervision of, Cees A. van der Voort. Horticultural examination of selected units initiated in May, 1994 has demonstrated that the combination of characteristics as herein disclosed for ‘Rousillon’ are firmly fixed and are retained through successive generations of asexual reproduction.

### BRIEF DESCRIPTION OF THE INVENTION

The following traits have been repeatedly observed and are determined to be basic characteristics of ‘Rousillon’ which in combination distinguish this lily as a new and distinct cultivar:

1. large flowers;

2. erect to vertical flowers arranged in a longitudinal axis;

3. red-purple color with contrasting orange pollen and marking and spots on the inner side of the throat; and

4. vigorous growth habit.

‘Rousillon’ has not been observed under all possible environmental conditions. The phenotype of the new cultivar may vary significantly with variations in environment such as temperature, light intensity, and daylength without any change in genotype. The following observations, measurements, and comparisons describe plants grown in the experimental research station CPRO-DLO, Wageningen, the Netherlands under conditions which approximate those generally used in commercial practice.

Of the many commercial cultivars known to the present inventor, no other cultivars are similar in comparison to ‘Rousillon’.

### BRIEF DESCRIPTION OF THE DRAWING

The accompanying color photographic illustration shows typical plant and flower characteristics of ‘Rousillon’, with colors being as true as possible with illustrations of this type.

### DETAILED BOTANICAL DESCRIPTION

In the following description, color references are made to The Royal Horticultural Society (RHS) Colour Chart. The color values were determined between 12 pm and 2 pm in June 1999 at the experimental research station CPRO-DLO, Wageningen, the Netherlands.

Origin: Selected crossing from a controlled hybridization program.

Parentage:

Female parent.—Unnamed seedling.

Male parent.—Unnamed seedling.

Classification:

Botanical.—A hybrid of the genus Lilium L.

Commercial.—Oriental Lily cv. ‘Rousillon’.

Plant:

Form.—A single stem carrying numerous, alternately arranged pedicels.

Height.—Short to medium for the mature plant, approximately 70 cm, depending on cultivation conditions.

Growth Habit.—Vigorous.

Stem.—Circular in transverse section; anthocyanin pigmentation present in spots and stripes; internodal length uniform.

Foliage:

Quantity.—Approximately 30-40, depending on cultivation conditions.

Size of leaf.—Approximately 8½ cm long and 3½ cm wide, depending on cultivation conditions.

Shape of leaf.—Generally ovate.

Texture.—Hardly any pubescence.

Color.—Light-green.

Bulb:

Size.—16 to 18 cm in circumference.

Color.—White/pink.

Bud:

Form.—Tapering like a lancet.

Size.—Short in length, depending on cultivation conditions.

Opening.—Unfolds normally in standard cultivation conditions.

Color.—Reddish-purple, RHS 60C, but more grayish just before opening.

Tepals.—Folded, three visible.

Flower:

Blooming habit.—Raceme.

Size.—Short to medium, approximately 14 cm, depending on cultivation conditions.

Borne.—Erect to vertical along a longitudinal axis.

Shape.—Generally, hexagonal star in form and bowl-shaped in cross-section.

Tepalage:

Number of Tepals.—6.

Arrangement.—Three inner and three outer.

Color:

Upper Surface.—Reddish-purple, between RHS 60B and RHS 60C.

Lower Surface.—Reddish-purple, RHS 64B.

Spotting or Marking.—Dark-purple spots, stripes and flakes are present on the inner side of the throat.

Pedicel:

Length.—Medium to large, depending on cultivation conditions.

Form.—Tapering like a lancet.

Texture.—Papillose.

Disease Resistance.—Medium.

Fragrance.—Present.

Lasting Quality.—Excellent.

Reproductive organs:

Stamens and anthers:

Arrangements.—Anthers form a generally circular pattern at their upper ends.

Number.—6.

Length.—Medium, depending on cultivation conditions.

Filaments Color.—Yellowish-green.

Pollen color.—Orange-brown.

Anther color.—Purplish-red.

Pistils.—Yellowish-green.

Stigma.—Gray, purplish fading.

Ovaries.—Triangular in shape.

I claim:

1. A new and distinct cultivar of Lily plant named ‘Rousillon’, as illustrated and described herein.
